(19) United States
(12) Patent Application Publication
(10) Pub. No.: US 2010/0034735 A1
(43) Pub. Date: Feb. 11, 2010

(54) TARGETED NANOPARTICLES FOR CANCER DIAGNOSIS AND TREATMENT

(76) Inventors: Jie CHEN, Edmonton (CA); Wilson Roa, Edmonton (CA)

Correspondence Address: THE NATH LAW GROUP, 112 South West Street, Alexandria, VA, 22314, US

(21) Appl. No.: 12/413,383

(22) Filed: Mar. 27, 2009

Related U.S. Application Data
(60) Provisional application No. 61/086,713, filed on Aug. 6, 2008.

(51) Int. Cl. A61K 51/00; A61K 49/00; A61K 9/16

(52) U.S. Cl. 424/1.29; 424/9.1; 424/490

(57) ABSTRACT

The invention provides modified gold nanoparticles that enable a non-invasive, real time, targeted cancer imaging-therapeutic in one step. After reaching the cancer targets, the designed targeted gold nanoparticles significantly enhance conventional treatment modalities at the cellular level. In this aspect the gold nanoparticles of the invention are modified to be bound to a Positron Emission Tomography (PET) tracer.

### CROSS-REFERENCE TO RELATED APPLICATIONS

This application claims the priority benefit of provisional application Ser. No. 61/086,713, filed on Aug. 6, 2008, which is incorporated herein by reference in its entirety.

### FIELD OF THE INVENTION

This disclosure relates to nanoparticles, and more particularly to targeted modified gold nanoparticles for diagnostic and therapeutic applications.

### BACKGROUND OF THE INVENTION

Throughout this application, various references are cited in parentheses to describe more fully the state of the art to which this invention pertains. Full bibliographic information for each citation is found at the end of the specification, immediately preceding the claims. The disclosure of these references is hereby incorporated by reference into the present disclosure in their entirety.

Various forms of radiation such as x-rays, laser light, and microwaves, as well as particle beams of, for example, neutrons, electrons, and protons, have been used to treat tumors. Unfortunately, such radiations are not generally very specific for the tumor and the dosages used often results in serious damage to normal tissue, thus limiting irradiation to lower doses that are not effective.

Radiosensitizer drugs are also utilized that act in combination with radiation to produce improved response, usually by making DNA more susceptible to radiation, or extending the life of free radicals produced by the radiation. Other radiation enhancers include elements or compounds that interact directly with the radiation to cause more tissue damage by increasing the absorption or scattering of the radiation, causing more local energy deposition by production of secondary electrons, alpha particles, Auger electrons, ionizations, fluorescent photons, and free radicals. For cancer therapy, the purpose is to selectively enhance dose to the tumor, so these drugs, elements or compounds must be preferentially accumulated in tumor tissue or the tumor tissue must respond in a preferential way, to spare the normal tissue.

Phytodynamic therapy (PDT) utilizes compounds that absorb visible light and result in formation of toxic free radicals. However, a disadvantage of this therapy is that it requires visible (laser) light to penetrate the tumor and is thus limited to superficial tissue, or those tissues that are optically accessible, generally superficial malignancies. Uniformity of dose delivery is also a problem due to the high absorbance of the light by tissue.

Boron Neutron Capture Therapy (BNCT) utilizes boron-10-containing compounds that have a high cross section for absorption of neutrons. Upon neutron capture, boron fissions into a lithium ion and alpha particle which have ranges of 5-9 microns and can locally damage DNA and kill cells. However, this method has several disadvantages for use.

Metals have been proposed for use in order to reduce cancerous growths. For example, U.S. Pat. No. 6,001,054 discloses a method for treating a site in a human body to inhibit abnormal proliferation of tissue at the site by introducing a metal surface at the site and then directing ionizing irradiation to the metal surface to obtain locally enhanced radiation therapy. Herold et al. (“Gold microspheres: a selective technique for producing biologically effective dose enhancement,” Int. J. Rad. Biol. 76: 1357-1364, 2000) discloses the use of gold particles with suspended living cells and with tumours during irradiation with x-rays. While a dose enhancement was found, no tumor remission or shrinkage in the animals was reported,

While the use of metal nanoparticles for cancer diagnosis and therapy has been contemplated, an effective means of how to deliver such nanoparticles to targeted cancer tissue is required. Various approaches for targeted delivery have been reported. For example, U.S. 2005/0020869 and U.S. 2005/0256360 disclose the use of gold nanoparticles for administration to enhance the effects of radiation therapy.

The advancement of cancer therapeutics, such as precise radiation therapy, refined chemotherapy/bio-molecular agents, as well as designer nanotechnology, has resulted in unprecedented cancer treatment potentials. Therapeutics in conjunction with traditional imaging studies that identify gross anatomy (e.g., computer-assisted tomography), and especially positron emission tomography (PET) has become an important clinical practice that detects metabolism and biology of cancer prior to gross tumor visibility.

It would be advantageous to provide a nanoparticle technology for cancer diagnosis and therapy that can be used in conjunction with imaging technology in an advantageous manner compared to that of the prior art.

### SUMMARY OF THE INVENTION

The present invention provides targeted modified gold nanoparticles (GNPs) wherein the modification to the GNPs leads to the effective targeting of the particles to a desired tissue for the provision of early diagnosis, imaging and/or treatment of diseases such as cancers. The present invention also includes the use of such GNPs in compositions and methods for the imaging, diagnosis and/or treatment of diseases.

In an aspect of the present invention is a modified gold nanoparticle comprising a gold core and a surface thereon, wherein said surface comprises a modification selected from a coating of cysteamine and/or cysteamine/thioglucose.

In an aspect of the present invention there are provided methods of eliminating tissue or cells by delivery of modified gold nanoparticles to the tissue or cells, then applying external energy that interacts with the modified gold nanoparticles.

In further aspects, the present invention provides methods of enhanced radiation therapy for promoting the shrinkage and/or elimination of tissues targeted for destruction by using targeted modified gold nanoparticles.

According to another aspect of the invention there are provided modified gold nanoparticles that enable a non-invasive, real time, targeted cancer imaging-therapeutic in one step. After reaching the cancer targets, the designer nanoparticles significantly enhance conventional treatment modalities at the cellular level. In this aspect the gold nanoparticles of the invention are modified to be bound to a Positron Emission Tomography (PET) tracer.

According to another aspect of the present invention are modified gold nanoparticles comprising a gold core and a surface thereon, wherein said modification comprises a coating of cysteamine or cysteamine/thioglucose on said surface. The coating may be provided as separate layers on said gold core.

According to an aspect of the present invention is a targeted modified gold nanoparticle comprising a gold nanoparticle bound to a PET tracer.

According to another aspect of the present invention is a targeted modified metal nanoparticle comprising a metal nanoparticle bound to a PET tracer.

According to another aspect of the present invention is a targeted modified gold alloy nanoparticle comprising a gold alloy nanoparticle bound to a PET tracer.

According to another aspect of the present invention is a targeted modified gold nanoparticle comprising a gold nanoparticle covalently bound to a PET tracer.

According to another aspect of the present invention is a targeted modified gold nanoparticle comprising a gold nanoparticle bound to a PET tracer, wherein said PET tracer is selected from an organic molecule labeled with a radionuclide selected from ¹¹carbon, ¹³nitrogen, ¹⁵oxygen and ¹⁸fluorine.

In aspects the PET tracer can be selected from [18F]flurodeoxyglucose and [18F]fluro-17-estradiol.

In aspects of the invention the modified gold nanoparticle comprises a gold nanoparticle, a hollowed gold nanoparticle, a gold nanoparticle having a functional molecule attached thereto, and a hollowed gold nanoparticle having a functional molecule attached thereto. In aspects the functional molecule is selected from cysteamine (AET) and thioglucose (Glu).

According to another aspect of the present invention there is provided a targeted modified gold nanoparticle comprising a gold nanoparticle covalently bound to a PET tracer, wherein said PET tracer is selected from [18F]flurodeoxyglucose and [18F]fluro-17-estradiol. In further aspects, the gold nanoparticle is hollowed and/or comprises AET and/or Glu.

According to a further aspect of the present invention is a composition comprising targeted modified gold nanoparticles.

According to another aspect of the present invention there is provided a composition comprising a gold nanoparticles covalently bound to a PET tracer, wherein said PET tracer is selected from [18F]flurodeoxyglucose and [18F]fluro-17-estradiol. In further aspects, the gold nanoparticle is hollowed and/or comprises AET and/or Glu.

According to another aspect of the present invention there is provided a method for real time targeted cancer imaging/therapy, said method comprising administering to a mammal diagnosed with a cancer, an effective amount of a modified gold nanoparticle covalently bound to a PET-tracer and subjecting said mammal to positron emission tomography for a time effective to visualize said cancer.

According to another aspect of the present invention is a method for enhancing the effects of radiation directed to a tissue or a population of cells in a mammal, comprising administering an amount of targeted modified gold nanoparticles to said mammal to achieve a concentration in said tissue or said population of cells of the mammal of at least about 0.1% metal by weight; and subsequently irradiating the mammal with radiation directed to said tissue or said population of cells, wherein said radiation is in the form of x-rays of about 1 keV to about 25,000 keV.

The present invention also provides methods for enhancing the effects of radiation directed to a tissue or cells in or from a mammal by administering an amount of targeted modified gold nanoparticles of the invention to the mammal or to the tissue or cells ex vivo, then irradiating the mammal with radiation directed to the tissue or cells, or irradiating the tissue or cells ex vivo. The methods of the present invention are useful for ablating unwanted tissues in a mammal without unacceptable damage to surrounding normal tissues or substantial toxicity to the mammal.

The present invention also provides methods for imaging tissue or cells in or from a mammal by administering an amount of targeted modified gold nanoparticles of the invention to the mammal or to the tissue or cells ex vivo, then applying PET scanning techniques to image the mammal or the tissue or cells ex vivo.

The present invention also provides methods for diagnosis and treatment of tissue or cells in or from a mammal by administering an amount of targeted modified gold nanoparticles of the invention to the mammal or to the tissue or cells ex vivo, then applying PET scanning techniques to image the mammal or the tissue or cells ex vivo for the purpose of diagnosis and/or treatment.

According to further aspects of the invention are methods of making the targeted modified gold nanoparticles of the invention.

According to yet another aspect of the invention is a method of making a targeted modified gold nanoparticle, wherein said method comprises;

covalently attaching a PET tracer to a gold nanoparticle, wherein said PET tracer is selected from an organic molecule labeled with a radionuclide selected from ¹¹carbon, ¹³nitrogen, ¹⁵oxygen and ¹⁸fluorine. In aspects of the method, the PET tracer can be selected from [18F]flurodeoxyglucose and [18F]fluro-17-estradiol. In further aspects of the method, the gold nanoparticle can have a having a functional molecule attached thereto. In aspects the functional molecule is selected from cysteamine (AET) and thioglucose (Glu).

In another aspect, the custom-designed gold nanoparticles (in stable binding with the PET-avid tracer) enable a non-invasive, real time, targeted cancer imaging-therapeutic in one step, by combining (i) enhanced imaging, (ii) radiotherapy, and (iii) real-time imaging in one single platform. In one embodiment, the PET-guided gold nanoparticles of the present invention combine [18F]flurodeoxyglucose for imaging and gold nanoparticles (GNPs) for treatment. After reaching the cancer targets, the designer nanoparticles significantly enhance conventional treatment modalities at the cellular level. When an external irradiation source, like X-ray, strikes the GNPs, radicals are generated to induce DNA damage. Due to synergistic multi-level targeting, the therapy can be effective in treating breast cancer with minimal in vivo side-effects to the normal tissue.

In another aspect, radiotherapy of breast cancer cells using the gold-based nanoparticles immuno-targeted to molecular markers on the cell surface is an effective modality to selectively kill cancer cells. The localized DNA damage can cause the rupture of the targeted cancer cell membranes and induce cell apoptosis. In MCF-7 breast adenocarcinoma cells, it engages the intrinsic pathway by enhancing upstream caspase activation. Radicals can be generated when X-ray strikes gold nanoparticles.

In another aspect, the present invention has the following advantages: 1) feedback information from pathology biomarkers can be used for the feature selection of functional targeting, 2) currently available 3D conformal radiotherapy planning can direct irradiation precisely at any depth inside body, and 3) synergistic radiation effect can be achieved at cellular level.

Other advantages of the present invention include:

i) Active and specific binding will significantly increase the local concentration of GNPs in cytoplasm. As a result, GNPs in the cytoplasm kill cancer cells more efficiently than those on the cell membrane and are a better choice for X-ray radiotherapy.

ii) Glu-GNPs enhance the radiation sensitivity in cancer cells, but not in nonmalignant cells. Therefore, lower irradiation dose is needed and thus reduce side effects of many cancer patients after radiotherapy.

iii) Synergistic cancer diagnosis and treatment can be achieved at a cellular level.

Other features and advantages of the present invention will become apparent from the following detailed description. It should be understood, however, that the detailed description and the specific examples while indicating embodiments of the invention are given by way of illustration only, since various changes and modifications within the spirit and scope of the invention will become apparent to those skilled in the art from said detailed description.

### DESCRIPTION OF THE FIGURES

The present invention will be further understood from the following description with reference to the Figures, in which:

FIGS. 1A and 1B. Gold nanoparticles: 1A. Electric micrograph of gold nanoparticles (Bar=50 nm); and 1B. the schematic diagram of naked gold nanoparticle binding with glucose.

FIG. 2. DU-145 cell uptakes of GNPs. Data are reported as the mean±SEM for three separate experiments performed in quadruplicate. The cell uptake values were (2.01±0.25)×104/cell for TGS-GNPs and (6.73±0.69)×104/cell for GNP-Glu. P<0.005 for GNP-Glu vs GNPs (t test).

FIG. 3 shows the distribution of GNPs in DU-145 cells.

FIG. 4. DU-145 cell growth was decreased by 13.52%, 17.82% and 14.72% after the treatment with TGS-GNPs, GNP-Glu, or 2 Gy X-ray at 24 h, respectively. Cell growth was not different after exposure to TGS-GNPs, GNP-Glu or 2 Gy X-ray treatment in vitro (P>0.1).

FIGS. 5A and 5B. Nanoparticles enhanced significantly radiosensitivity of DU-145 cells. Irradiation of 2Gy alone induced the inhibition rate of cell growth (15.78+2.85)% after 24 hours and (19.03+6.00)% after 48 hours. A. Irradiation of 2Gy+TGS-GNPs were (30.57+3.32)% at 24 hours and (32.18+2.12)% at 48 hours. B. Irradiation of 2Gy+Glu-GNPs were (45.97+3.95)% at 24 hours and (44.63+1.87)% at 48 hours. Both TGS-GNPs and Glu-GNPs can increase radiosensitivity of 2Gy (P<0.001) X-ray on DU-145 cells after 24 and 48 hours.

FIG. 6. Comparison of radiosensitivity enhancements of between DU-145 cells induced by Glu-GNPs and those induced by TGS-GNPs. The combination of Glu-GNP with X-ray induced an increased inhibition rate of 50.3% and 38.7% compared with GNPs only after 24 h (P<0.005), and 48 h (P<0.001) respectively.

FIG. 7. Schematic diagram of synthesis of thioglucose-capped gold nanoparticle (Glu-Capped GNPs), and Cysteamine-capped gold nanoparticles (AET-capped GNPs).

FIG. 8. (A) TEM and (B, C) HRTEM images of GNPs with the diameter about 10.8 nm (refer to inset A). The size distribution of GNPs was determined by DLS.

FIG. 9. XPS results of (A) AET-GNPs and (B) Glu-GNPs.

FIG. 10. TEM images of MCF-7 cell uptakes for GNPs, the targeted cells were treated with or without GNPs for two hours: (A) Control cell; (B) AET gold nanoparticles; and (C) Glucose gold nanoparticles. Left bottom subfigure shows an enlarged picture of the part in FIG. 10 (C).

FIG. 11. Cell uptakes of GNPs bound with either AET or thioglucose.

FIG. 12. Toxicity test of AET-GNP, Glu-GNP and control in MCF-7 cells by MTT assay.

FIG. 13. (A) The cytotoxicity on MCF-7 breast cancer cells induced by 200 kVp X-ray irradiation with or without gold nanoparticles at 48 hours. (B) Cell survival rate determined by clonogenic survival assay (at 5 days and 14 days).

FIG. 14. A. Comparison of radiation sensitivity in cancers and non-malignant cells. B. Comparison of the cytotoxicity induced in MCF-7 cell line by various types irradiation with gold nanoparticles (48 hours after irradiation).

FIG. 15 shows the schematics of PET-guided Gold Bullet and the [18F]flurodeoxyglucose molecule.

FIG. 16. (A) TEM and (B, C) High-resolution TEM images of GNPs with the diameter about 10.8 nm. (insert of A). The size distribution of GNPs determined by DLS.

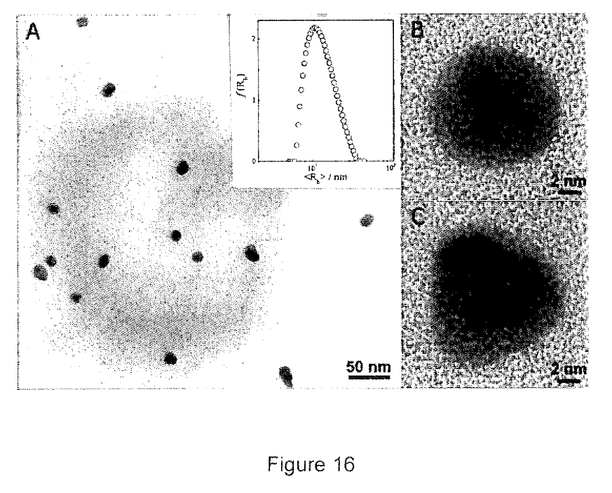

FIG. 17. NMR spectrum to validate the covalent binding between nanoparticles and PET tracers. Here Ac (Acetyl) is a functional group and is used to protect the binding site. AcO will is replaced by OH group when it is to be used as FDG.

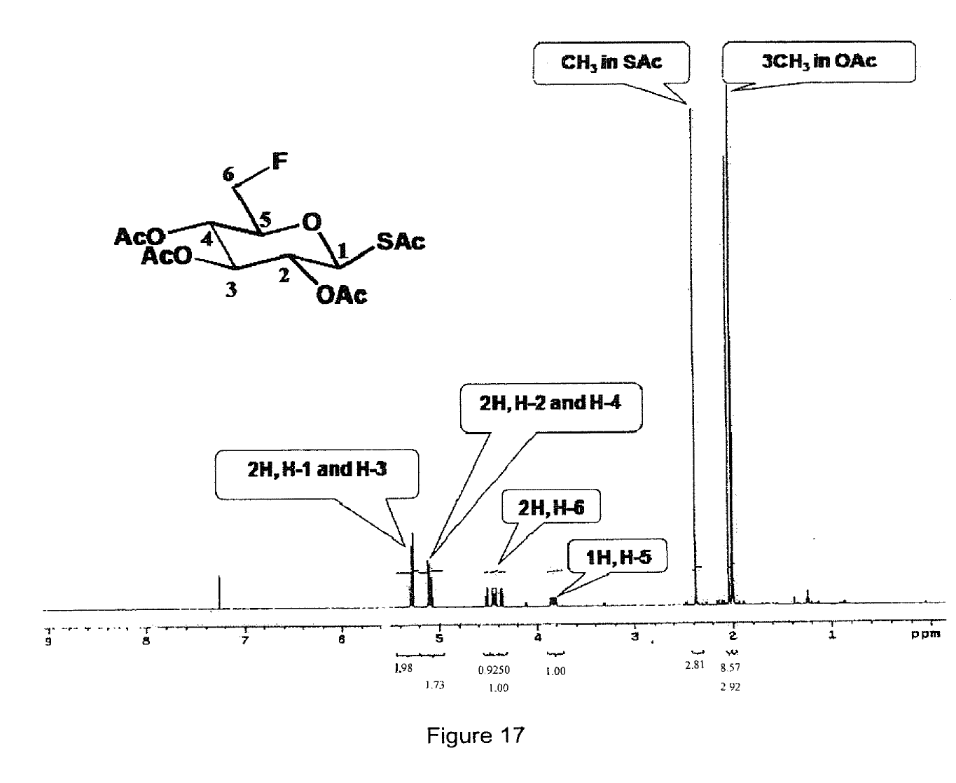

FIG. 18. TEM images of MCF-7 cell uptakes for GNPs, the targeted cells were treated with or without GNPs for two hours: (A) Control cell; and (C) Glucose gold nanoparticles. Left bottom subfigure shows an enlarged picture of the part in FIG. 18(C).

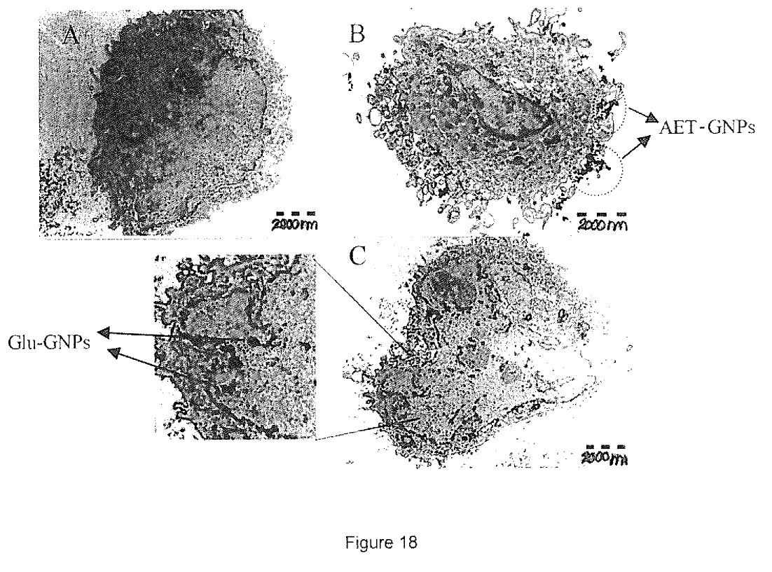

FIG. 19. Comparison of MCF-7 cell tumour volume in mice treated with control (-▪-); FDG-GNPs (--); irradiation only (-▴-); and irradiation plus FDG-GNPs (-▾-).

### DETAILED DESCRIPTION OF THE INVENTION

Unless defined otherwise, all technical and scientific terms used herein have the meaning commonly understood by a person skilled in the art to which this invention belongs. As used herein, the following terms have the meanings ascribed to them unless specified otherwise.

As used herein, “positron emission tomography imaging (PET)” incorporates all positron emission tomography imaging systems or equivalents and all devices capable of positron emission tomography imaging. The methods of the invention can be practiced using any such device, or variation of a PET device or equivalent, or in conjunction with any known PET methodology. See, e.g., U.S. Pat. Nos. 6,151,377; 6,072,177; 5,900,636; 5,608,221; 5,532,489; 5,272,343; 5,103,098. Animal imaging modalities are included, e.g. micro-PETs (Corcorde Microsystems, Inc.).

As used herein, external energy, e.g., radiation, is directed to a tissue or a population of cells targeted for destruction or ablation, also referred herein as a “target tissue” or “target cells”. By “ablating a tissue or cell” is meant that the growth of the tissue or cell is inhibited, the size of the tissue or the number of the cells is reduced, or the tissue or cell(s) is eliminated. By administering metal nanoparticles to a mammal, or to tissues or cells ex vivo, the therapeutic effects of external energy or radiation is enhanced by way of the interaction of the particles with the sources of energy or radiation, resulting in an increased energy deposition in the vicinity of the particles. This is also referred to herein as “Metal Enhanced Radiation Therapy”, or “MERT”. Metal-Enhanced Radiation Therapy (MERT) is able to kill unwanted tissue or cells in a highly specific manner.

As used herein, “enhanced radiation therapy” or “enhancing the therapeutic effects of radiation” is meant that a lower dose of radiation is required to achieve efficacy (e.g., the target tissue is ablated or eliminated) with metal nanoparticles as compared to without metal nanoparticles; or, better efficacies are achieved by a given dose of irradiation with metal nanoparticles as compared to without metal nanoparticles.

### Targeted Gold Nanoparticles

The present invention provides gold nanoparticles (GNPs) for use in the imaging, diagnosis and/or treatment of disorders in vivo and ex vivo. The gold nanoparticles may be modified with different functional molecules such that they target cells in vivo or ex vivo in mammals and mammalian tissues. The gold nanoparticles may also be modified to be hollow in order to encapsulate a drug and act as a controlled release drug carrier. The gold nanoparticles of the invention may also further be bound to a PET-tracer to enable cancer imaging and therapeutics with radiation and in particular, the targeted gold nanoparticles of the invention are used to image, diagnose and treat various forms of cancers in a mammal.

Modifications to the GNPs of the invention include but are not limited to providing different functional molecules as a coating thereon. The functional molecules may be selected from a type of amine coating such as cysteamine (AET), thioglucose (Glu) and combinations thereof. As modified using such functional amines, the gold nanoparticles may bind to the outside of cells (cysteamine) or intracellularly (Glu). AET-capped GNPs are strongly positive and selectively bind onto the cell's surface. Glu-capped GNPs target the cell cytoplasm and take advantage of the fact that cancer cells have an increased requirement for glucose. Such modified GNPs enhances radiation cytotoxicity and provides a more effective cancer treatment.

Radiation can be in the form of low energy (about 1 to 400 KeV) or high energy x-ray (about 400 KeV up to 25,000 KeV). Radiation can also be in the form of microbeam arrays of x-ray or radioisotopes. Other forms of radiation suitable for use in practicing the methods of the present invention include, but are not limited to, visible light, lasers, infrared, microwave, radio frequencies, ultraviolet radiation, and other electromagnetic radiation at various frequencies. Various sources or forms of radiation can be combined, particularly for treating tumors at depth.

Enhanced radiation therapy may be applied to a human to destroy unwanted tissue, e.g., a tumor. The modified gold nanoparticles can be administered to human (or animal) prior to irradiation by standard methods, e.g., intravenous or intra-arterial injection, direct injection into a target tissue (e.g., tumor), and implantation of a reservoir device capable of a slow release of the modified gold nanoparticles. In general, nanoparticles are administered in an amount to achieve a concentration in the human/animal of at least about 0.05 to 10% gold by weight, preferably 0.1 to 5% gold by weight, and more preferably 0.3% to 2% gold by weight, in order to achieve radiation enhancement. The enhanced radiation therapy may be applied to tissues or cells ex vivo to ablate or destroy unwanted tissues or cells from a human/animal. For example, the enhanced radiation methods can be applied to bone marrow ex vivo to eliminate unwanted cells prior to transplantation, or applied to a donor organ to remove immunogenic cells prior to transplantation.

The enhanced radiotherapy methods disclosed herein can be used in conjunction with other existing therapies, such as chemotherapy, anti-angiogenesis therapy, boron neutron capture therapy (or BNCT) and other drug therapy.

The nanoparticles disclosed herein can also be used as drug delivery agents. Due to the size of the nanoparticles, the use of nanoparticles to deliver drugs to the brain across the blood-brain barrier (BBB) is possible. An advantage of nanoparticle carrier technology is that nanoparticles mask the blood-brain barrier limiting characteristics of the therapeutic drug molecule. The system may slow drug release in the brain, decreasing peripheral toxicity. Thus, the nanoparticles disclosed herein can be used in non-limiting aspects to treat Central Nervous System (CNS) diseases. The PET-guided nanoparticles can also access brain metastasis in cancers.

In non-limiting embodiments of the invention, modified gold nanoparticles (GNPs) were developed and synthesized with two kinds of functional molecules: Cysteamine (AET) and thioglucose (Glu). Their cell uptake and radiation cytotoxicity enhancement in a breast cancer cell line (MCF-7) versus a non-malignant breast cell line (MCF-10A). Transmission Electron Microscopy (TEM) results showed that cancer cells take up functional Glu-GNPs significantly more than naked GNPs. The TEM results also indicated that AET-capped GNPs are mostly bound to the MCF-7 cell membrane, while Glu-GNPs enter the cells and are distributed in the cytoplasm. After MCF-7 cell uptake of Glu-GNPs, the in vitro cytotoxicity effects were observed at 24, 48, and 72 hours. The results showed that these functional GNPs have little or no toxicity to these cells. To validate the enhanced killing effect on cancer cells, we have applied various forms of radiation, such as 200 kVp X-rays and γ-rays, to the cells, both with and without functional GNPs. By comparison with irradiation alone, the results showed that GNPs significantly enhanced cancer killing.

### Prostate Cancer—Modified Gold Nanoparticles Enhanced Radiation Sensitivity in Prostate Cancer Cells

Gold nanoparticles (GNP) were made and used to enhance radiation sensitivity and growth inhibition in radiation-resistant human prostate cancer cells. Gold nanoparticles (GNPs) were synthesized as described herein in Example One. Exposure to Glu-GNPs resulted in a three times increase of nanoparticle uptake compared to that of TGS-GNPs in each target cell (p<0.005). Cytoplasmic intracellular uptake of both TGS-GNPs and Glu-GNPs resulted in a growth inhibition by 30.57% and 45.97% respectively, comparing to 15.88% induced by irradiation alone, in prostate cancer cells after exposure to the irradiation. GluGNPs showed a greater enhancement, 1.5 to 2 fold increases within 72 hours, on irradiation cytotoxicity compared to TGS-GNPs. Tumor killing, however, did not appear to correlate linearly with nanoparticle uptake concentrations. These results showed that functional glucose-bound gold nanoparticles enhanced radiation sensitivity and toxicity in prostate cancer cells.

### Breast Cancer—Modified Gold Nanoparticles Enhanced Radiation Cytotoxicity in Breast Cancer Cells

Gold nanoparticles (GNPs) and modified GNPs with two kinds of functional molecules: Cysteamine (AET) and thioglucose (Glu) were synthesized and demonstrated their cell uptake and radiation cytotoxicity enhancement in a breast cancer cell line (MCF-7) versus a nonmalignant breast cell line (MCF-10A). Transmission Electron Microscopy (TEM) results showed that cancer cells take up functional Glu-GNPs significantly more than naked GNPs. The TEM results also indicated that AET-capped GNPs were mostly bound to the MCF-7 cell membrane, while GluGNPs enter the cells and are distributed in the cytoplasm. After MCF-7 cell uptake of Glu-GNPs, or binding of AET-GNPs, the in vitro cytotoxicity effects were observed at 24, 48, and 72 hours, respectively. The results showed that these functional GNPs have little or no toxicity to these cells. To validate the enhanced killing effect on cancer cells, various forms of radiation, such as 200 kVp X-rays and y-rays, to the cells, both with and without functional GNPs. By comparison with irradiation alone, these results showed that GNPs significantly enhanced cancer killing.

As described above gold nanoparticles can be made to be solid or hollow to contain a desired therapeutic drug. The gold nanoparticles can also be made to have different functional molecules as a coating thereon as is described herein. The functional molecules can be coated on gold nanoparticles that are either solid or hollow. If hollow the gold nanoparticles can be provided with the functional molecules as a coating and also contain a drug within the gold nanoparticle.

The gold nanoparticles of the invention for use in radiation therapy are composed of a metal core and provided with a modified surface layer surrounding the metal core. The metal core in many aspects of the invention is gold. However, it is also understood by one of skill in the art that the metal may be made of a gold alloy or other metal altogether. Metals which can be used to form the modified targeted nanoparticles of the invention for enhancing radiation effects are heavy metals, or metal with a high Z number, including but not limited to gold, silver, platinum, palladium, cobalt, iron, copper, tin, tantalum, vanadium, molybdenum, tungsten, osmium, iridium, rhenium, hafnium, thallium, lead, bismuth, gadolinium, dysprosium, holmium, and uranium. The preferred metal is gold in an aspect. The metal core can consist of one metal, or it can be a mixture or an ordered, concentric layering of such metals, or a combination of mixtures and layers of such metals.

The size of the gold core may be from about 0.8 up to about 400 nm in diameter. The gold nanoparticles of this size selectively increase the local radiation dose directed to a target tissue such as a tumor and do not exhibit substantial toxicity in the mammal. Preferably, the core is of a size in the range of at least about 0.8 or 1 to 50 nm; more preferably, about 0.8 or 1 to 20 nm in diameter; and even more preferably, about 0.8 to 3 nm diameter. Especially preferred size of the metal core is up to about 10-11 nm in diameter.

In all embodiments of the gold nanoparticles described herein, the modified gold nanoparticle may be bound to a PET tracer in order to be used for early cancer diagnosis and the modification may be used effectively to provide therapy. In aspects, this is covalent binding. The PET tracer may be selected from an organic molecule labeled with a radionuclide selected from ¹¹carbon, ¹³nitrogen, ¹⁵oxygen and ¹⁸fluorine. In aspects the PET tracer can be selected from [18F]flurodeoxyglucose and [18F]fluro-17-estradiol.

In one non-limiting embodiment of the present invention the gold nanoparticle of the invention is covalently bound to [18F]flurodeoxyglucose and is used for early cancer diagnosis. In aspects the cancer is breast cancer and prostate cancer. Because of the flurodeoxyglucose, the gold nanoparticles can be specifically targeted at tumor cells but not normal cells.

In aspects of the invention the tissues or cells targeted for destruction are cancerous (i.e. tumors) and include any solid tumors such as carcinomas, brain tumor, melanomas, lymphomas, plasmocytoma, sarcoma, glioma, thymoma, and the like. The present invention can also be used to treat tumors of the oral cavity and pharynx, digestive system, respiratory system, bones and joints, soft tissue, skin, breast, genital system, urinary system, prostate, eye and orbit, brain and other nervous system, endocrine system, myeloma, and leukemia.

### Formulation and Administration Pharmaceuticals

The invention provides pharmaceutical formulations comprising the targeted modified gold nanoparticles (GNPs) of the invention and a pharmaceutically acceptable excipient suitable for administration an imaging enhancing agents, and methods for making and using these compositions. These pharmaceuticals can be administered by any means in any appropriate formulation. Routine means to determine drug regimens and formulations to practice the methods of the invention are well described in the patent and scientific literature. For example, details on techniques for formulation, dosages, administration and the like are described in, e.g., the latest edition of Remington's Pharmaceutical Sciences, Maack Publishing Co, Easton Pa. As used herein, a pharmaceutically acceptable carrier includes solvents, dispersion media, isotonic agents and the like. Examples of carriers include water, saline solutions, sugar, gel, porous matrices, preservatives and the like, or combinations thereof.

The modified gold nanoparticles of the invention may be administered to an animal by standard methods, including but not limited to intravenous or intra-arterial injection, direct injection into a target tissue (e.g., tumor), and implantation of a reservoir device or cavity capable of a slow release of metal nanoparticles. Intravenous injection is well suited to delivery of gold nanoparticles to the vascular system of an animal, and is a preferred method of administration where the target tissue to be ablated is a tumor.

Direct intratumoral or tissue injection is also possible in order to reduce the concentration of metal nanoparticles in other tissues and achieve a high concentration in the tumor or tissue to be ablated. Use of small gold nanoparticles, e.g., 0.8 to 10 nm in diameter, facilitates diffusion of the particles throughout the parenchyma of the targeted tissue. Linking nanoparticles to a specific targeting molecule (i.e. PET tracer) can facilitate localization of the nanoparticles to specific cells, as disclosed hereinabove.

A reservoir of the modified gold nanoparticles can be provided adjacent to or in the bed of a target tissue. The reservoir can be a permeable bag or container, or a wafer, string, gel, Matrigel, or other material preloaded with the nanoparticles, or a time release pump driven osmotically, mechanically, or electrically. The modified gold nanoparticles can be delivered over a controlled time period, and longer exposure to the nanoparticles may be achieved as compared to administration by intravenous injection.

The pharmaceutical formulations of the invention comprising the targeted modified gold nanoparticles (not having the PET tracer) can be presented in unit-dose or multi-dose sealed containers, such as ampoules and vials, and can be stored in a freeze-dried (lyophilized) condition requiring the addition of the sterile liquid excipient, for example, water, for injections, immediately prior to use. Extemporaneous injection solutions and suspensions can be prepared from sterile powders, granules, and tablets.

It is understood by one of skill in the art that the targeted modified gold nanoparticles of the invention can be provided in a formulation in a combination of differently made nanoparticles. That is a combination of AET-coated GNPs and Glu-coated GNPs and/or GNPs-PET tracer can be used together in a formulation or used concurrently as desired.

### Kits

The invention provides kits comprising the targeted modified gold nanoparticles of the invention and compositions containing such. The kits also can contain instructional material teaching methodologies, e.g., how and when to administer the pharmaceutical compositions, how to apply the compositions and methods of the invention to imaging systems, e.g., computer assisted tomography (CAT), magnetic resonance spectroscopy (MRS), magnetic resonance imaging (MRI), positron emission tomography (PET) or single-photon emission computed tomography (SPECT). Kits containing pharmaceutical preparations may include directions as to indications, dosages, routes and methods of administration, and the like.

The above disclosure generally describes the present invention. A more complete understanding can be obtained by reference to the following specific Examples. These Examples are described solely for purposes of illustration and are not intended to limit the scope of the invention. Changes in form and substitution of equivalents are contemplated as circumstances may suggest or render expedient. Although specific terms have been employed herein, such terms are intended in a descriptive sense and not for purposes of limitation.

### EXAMPLES

### Example One

### Enhanced Radiation Sensitivity in Prostate Cancer by Gold-Nanoparticles

### Chemicals

All chemicals were obtained from Sigma-Aldrich (Milwaukee, Wis.). MTT CellTiter 96 non-radioactive cell proliferation assay kit was purchased from Promega (Madison, Wis.).

### Synthesis of Gold Nanoparticles

The general synthesis method for making gold nano-particles follows three substeps. i) 3.2 ml of 25 mM HAuCl₄ solution was added into 60 ml of deionized water in an ice bath with moderate stirring. ii) 4 ml of 26 mM NaBH4 was then added as a reductant to obtain naked gold nanoparticles. iii) The naked GNPs solution was added into two tubes each containing 22.4 ml of naked GNPs solution. 4 ml of 20 mM 1-thio-13-glucose or 4 ml of 38.8 mM sodium citrate solution was added separately into two gold solutions.

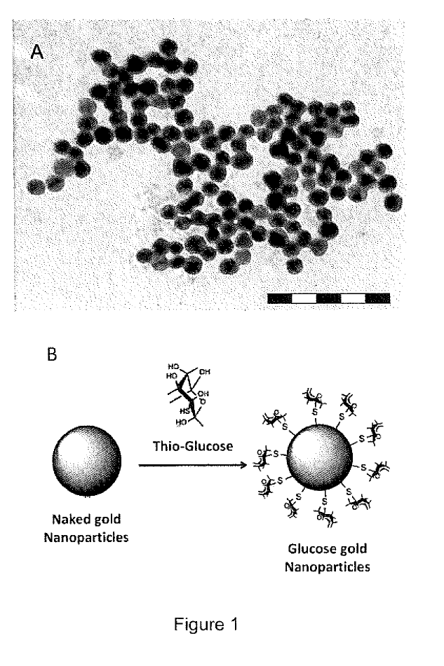

Thio-glucose covalently and sodium citrate electrostaticly bind to the GNPs to form functionalized thioglucose-capped gold nanoparticles (Glu-GNPs) and neutral gold nanoparticles (TGS-GNPs) respectively. (FIGS. 1A and 1B). Both the TGS-GNPs and the Glu-GNPs were dialysed for two days to remove any free sodium citrate or thio-glucose from the gold particle solutions before these solutions were provided to the experiments. Both the TGS-GNPs and the Glu-GNPs were characterized using transmission electronic microscopy (TEM), 1CP-MS and Kratos Axis 165 X-ray Photoelectron Spectroscopy (XPS) (Kratos Analytical) as described previously.”

### Cell Culture

Human prostate carcinoma cell line DU-145 was used in all the experiments. DU-145 cell line was purchased from the American Type Culture Collection (Manassas, Va., USA). The cells were maintained in Dulbecco's modified Eagle's medium supplemented with 10% FBS, 20 mM D-glucose, 100 Ul/ml penicillin G, and 100 gg/ml streptomycin in a humidified incubator with 5% CO₂ in the air at 37° C. DMEM without glucose was used for the cells that were exposed to either Glu-GNPs or Glu-GNPs plus Cytochalasin B (glucose transport inhibitor).

### Uptake Assay

The assay was performed in triplicate. A 10 ml DU145 cell suspension containing 2×10⁶ cells was seeded onto a 100 mm-cell culture dish and was cultured overnight. When the cells reached a 70% confluence, the target cells were exposed to the vehicle, 15 nM TGS-GNPs, or 15 nM Glu-GNPs, respectively at 37° C. After two hours of incubation, the free GNPs in the cell cultures were removed by washing the cells twice with the PBS buffer. The cells were detached with Trypsin-EDTA. After centrifugation and the removal of the supernatant, the cells were resuspended in the PBS with a final volume of 5 ml. The number of cells in suspension was counted with a hemocytometer. 5 ml of 50% HNO₃ was added to each sample to lyse the cells. The gold mass in the lysis solution was measured using Inductively Coupled Plasma Mass Spectrometry (ICP-MS). The number of gold nanoparticles was calculated via the gold mass, and the number of GNPs in the lysis solution was divided by the number of cells to yield the number of GNPs taken up by cells.

### Transmission Electron Microscopy

The cells treated with or without GNPs were collected by centrifugation. The cell pellets were fixed in 4% (v/v) formaldehyde in 0.1 M phosphate buffer (pH 7.2) for four hours at 4° C. After being washed in the same buffer, the cells were resuspended in 1% 0s04 for one hour at room temperature. They were then washed twice by centrifugation and resuspended in distilled water. The final pellet was resuspended in a small volume of warm 2% (w/v) agarose, poured onto a glass slide, and allowed to cool. When set, the small pieces of gel containing the cells were cut out and dehydrated through a graded series of ethanol solutions. The pieces were then embedded in epoxy resin, and thin sections were cut with an ultramicrotome, stained with uranyl acetate followed by lead citrate and examined in Philips EM301 electron microscope operating at 80 kV.

### Irradiation of Cells

All cell irradiation treatments were carried out using a Pautak Therapax 3 Orthovoltage 244 Monitor Units/minute X-ray machine at 200 kVp using a 0.35 CU+1.5 AL filter. DU145 cells in 100-mm culture dishes were irradiated at room temperature when cultures reached 75% confluence. Cells received either a mock treatment for control or 2 Gy. After irradiation, cultures were returned to 5% CO₂/37° C. incubation until they were harvested at the time points required.

### MTT Assay

MTT assay is a quantitative colorimetric method to determine cytotoxicity. It utilizes the yellow tetrazolium salt [3-(4,5-Dimethylthiazol-2-yl)-2,5-diphenyltetrazolium-bromide] which is metabolized by mitochondrial succinic dehydrogenase activity of proliferating cells to yield a purple formazan reaction product. In the present study, toxicity in mitochondria induced by GNPs with or without radiation was measured by an MTT assay in 96-well plates. The experiments were performed by eight replicates and cells were seeded in 96-well plates (3×10³/well) with 200 IA of culture medium per well. The cells were allowed to grow on the 96-well plates overnight. Cells were then exposed to either TGS-GNPs or Glu-GNPs respectively and different doses of irradiation according to the experimental design. The cell responses were monitored by the MTT assay by following the manufacturer's instructions. After removal of the medium, 100 μl of MTT (0.4 mg/ml) dissolved in medium were added to each well. Following three hours of incubation, the medium was replaced with 100 of 0.1 N HCl/isopropanol, and absorbance in each well was assessed at 550 nm using a microplate reader. Absorbance was expressed as a percentage of control. The cell growth inhibitory rate was calculated by the following formula: Inhibitory rate=(1−average OD₅₅₀ₙₘ of treated group/average OD₅₅₀ₙₘ of control group)×100%.

### Statistical Analysis

In the statistical analysis, differences between the treated and control groups were compared using Student's t-tests, with the differences at the P<0.05 level considered to be statistically significant.

### Uptake of GNPs on DU-145 Cells

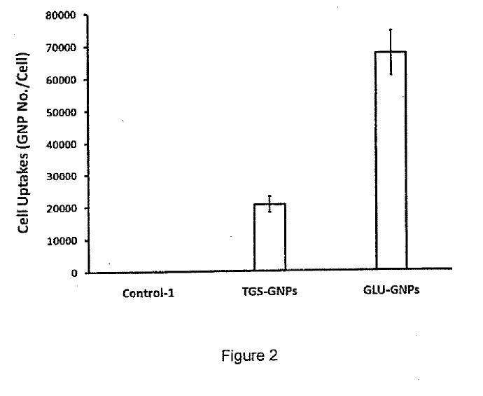

After exposure to 15 nM naked TGS-GNPs and Glu-GNPs respectively for two hours, the average number of GNPs per cell associated with each DU-145 cell was (2.06±0.24)×10⁴ for TGS-GNPs, and (6.73±0.67)×10⁴ for Glu-GNPs (FIG. 2). In contrast, exposure to Glu-GNPs results in a three times increase of nanoparticle uptake compared to TGS-GNPs in each target cell (FIG. 2). P<0.005 for Glu-GNPs vs. TGS-GNPs (t test).

### Distribution of Glu-GNPs in DU-145 Cells

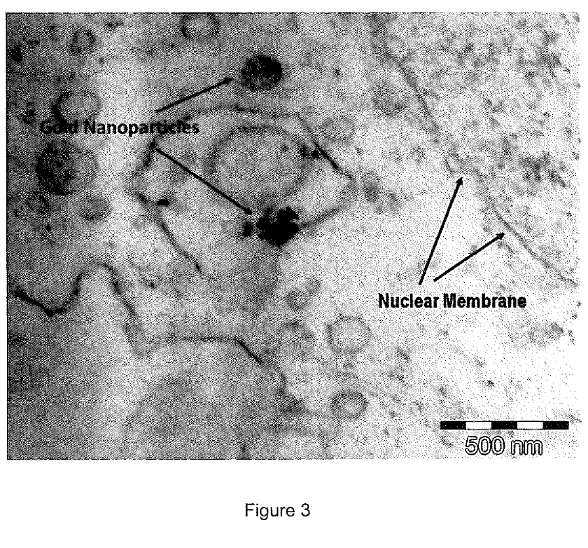

The distribution of GNPs in DU-145 cells was determined by TEM. FIG. 3 is the micrograph of the cells treated with 15 nM Glu-GNPs. The figure shows that most of Glu-GNPs were distributed in the cytoplasm.

### Effect of Gold-Particles on DU-145 Cell Growth

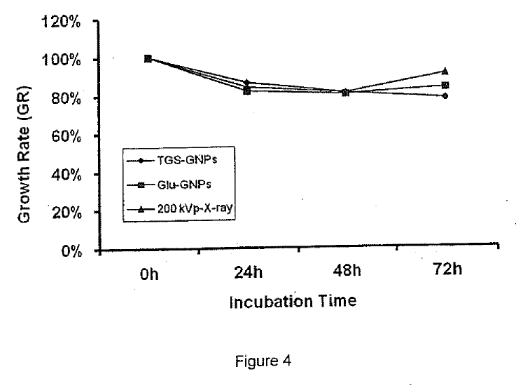

Compared to the control, the results in FIG. 4 show that DU-145 cell growth was decreased by 13.52% with TGS-GNPs and 17.82% with Glu-GNP treatments (P<0.01) after 24 hours. However, there was no difference on cell growth between the TGS-GNPs and Glu-GNPs.

### Effect of 2 Gy 200 kVp X-ray on DU-145 Cell Viability

The cytotoxic effects of X-ray on DU-145 cells were analyzed after 24, 48 and 72 hours of irradiation. Untreated control samples were arbitrarily assigned a value of 100% and the results of all treatments were normalized to 100% (i.e., % of control). The data in FIG. 4 shows that 2 Gy X-ray induced an inhibition of cell growth by 15.78%, 19.03% and 9.22% at 24, 48 and 72 hours respectively.

### Gold-Particles Enhance Radiation Cytotoxicity on DU-145 Cell

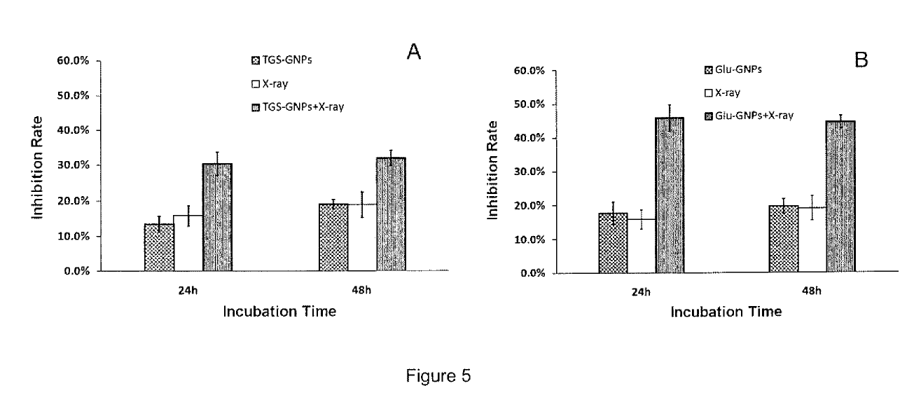

To determine whether GNPs had enhanced radio sensitivity of DU 145 cells to 2 Gy X-ray, cells were treated with either a single dose of 2Gy X-ray or 2Gy X-ray and GNPs, whereas control group did not receive any treatment. Untreated control samples were arbitrarily assigned a value of 100% and the results of all treatments were normalized to 100% (i.e., % of control). FIG. 5A shows that either TGS-GNPs or X-ray induced an inhibition of cell growth by 13.52% or 15.88% at 24 h, individually. However, a combination of TGS-GNPs and X-ray induced an inhibition of cell growth of 30.57% (P<0.005) (FIG. 5A). Similarly, the data in FIG. 5B shows that Glu-GNPs induced an inhibition of cell growth by 17.82% after 24 hours but the combination of Glu-GNPs plus X-ray induced an inhibition of cell growth by 45.97% (P<0.005).

### Enhancement of Radiosensitivity by Either TGS-GNPs or Glu-GNPs

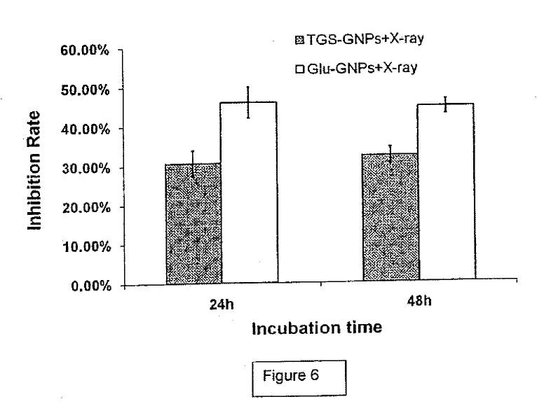

To evaluate whether glucose will help the delivery of gold-nanoparticles to cancer cells, the cellular uptakes (FIG. 2) and radiosensitivity enhancement induced by Glu-GNPs were determined and compared to those induced by TGS-GNPs. FIG. 6 shows that the inhibition rate of 2Gy X-ray plus TGS-GNPs was (30.57±3.32)% at 24 hours and (32.18±2.12)% at 48 hours. The inhibition rate of 2Gy X-ray plus Glu-GNPs was (45.97±3.95)% at 24 hours and (44.63±1.87)% at 48 hours. Glu-GNPs increased radiosensitivity by 50.37% (P<0.001) at 24 hours and 38.68% (P<0.005) at 48 hours compared with TGS-GNPs that have no glucose bound.

### Example Two

### Enhancement of Radiation Cytotoxicity in Breast Cancer Cells by Localized Attachment of Gold Nanoparticles

### Materials

All chemicals were obtained from Sigma-Aldrich (Milwaukee, Wis.). MTT cell proliferation assay kit was purchased from Invitrogen (Burlington, Ontario).

### Synthesis of Gold Nanoparticles

The general synthesis method for making gold nanoparticles followed three sub-steps. i) 2 ml of 25 mM HAuCl₄ solution was added into 25 ml of deionized water in an ice bath with moderate stirring. ii) 2 ml of 30 mM NaBH₄ was then added as a reductant to obtain GNPs without any capping agents. iii) To functionalize the GNPs, 4 ml of 25 mM thio-glucose or AET was added into the previous gold solution, respectively, to obtain functional gold nanoparticles. Considering the gold nanoparticles in step (ii) are easy to aggregate, sodium citrate (TGS) was added to cap them as naked gold nanoparticles. The purpose for using the same GNP solution was to ensure the resulting functionalized nanoparticles had identical sizes. Both AET-capped gold nanoparticles (AET-GNPs) and thioglucose capped gold nanoparticles (Glu-GNPs) were dialysed for two days before cell uptake and irradiation experiments. The average sizes of three types of GNPs (naked GNPs, AET-GNPs and Glu-GNPs) measured by dynamic light scattering (DLS) were about 10.8 nm. The surface of AET-GNPs and GluGNPs are characterized by using X-ray Photoelectron Spectroscopy (XPS) (Kratos Analytical).

### Cell Culture and Culture Conditions

Human breast adenocarcinoma line MCF-7 was used in all the experiments. MCF-7 cell line was purchased from American Type Culture Collection (Manassas, Va., USA). MCF-7 cells were maintained in Dulbecco's Modified Eagle Medium (DMEM, Invitrogen) containing 10% FBS (Invitrogen), 100 UI penicillin G and 100 μg/ml streptomycin (Sigma). The cells were incubated at 37° C. in a humidified 5% CO₂ atmosphere.

Cellular Morphology with Transmission Electron Microscopy

The cell cultures both treated with and without GNPs for two hours were centrifuged and the supernatants were removed. The pellets with 2 PBS washes were fixed in 4% (v/v) formaldehyde in 0.1 M phosphate buffer (pH 7.2), for two hours at 4° C. After being washed in the same buffer, the cells were resuspended in 1% 0s0₄ for one hour at room temperature. They were then washed twice by centrifugation and resuspension in distilled water. The final pellet was resuspended in a small volume of warm 2% (w/v) agarose, poured onto a glass slide, and allowed to cool. When set, small pieces of gel containing the cells were cut out and dehydrated through a graded series of ethanol solutions. The pieces were then embedded in epoxy resin, and thin sections were cut with an ultramicrotome, stained with uranyl acetate followed by lead citrate and examined in a Philips EM301 electron microscope operating at 80 kV.

### Determination of GNPs Bound to or Uptaken by Target Cells

A 5 ml MCF-7 cell suspension containing 5×10⁵ cells was added to 6 cm-dishes and cultured overnight. When the cells reached 70% confluence, the target cells were exposed to 5 ml of fresh medium with 15 nM of GNPs (final concentration). After two hours of incubation, the medium with GNPs was removed and cells were washed twice with 5 ml PBS buffer. The cells were detached with Trypsin/EDTA. After centrifugation and removal of the supernatant, the cells were resuspended into PBS to a final volume of 5 ml. The number of cells in suspension was counted with a hemocytometer. 5 ml of 20% HNO₃ was added into each sample to lyse the cells. The gold mass in the lysis solution was measured by Inductively Coupled Plasma Mass Spectrometry (ICP-MS). We can calculate the number of gold nanoparticles via the gold mass, and the number of GNPs in the lysis solution divided by the number of cells provided a quantitative measure of GNP cell uptake.

### MTT Assay and Clonogenic Survival Assay

The cytotoxicity induced by GNPs with and without radiation was assessed by an MTT assay in 96-well plates and clonogenic survival assay. MCF-7 cells were tested. The starting number of cells was 3000 per well with 150 μl of medium. The cells were allowed to grow on the 96-well plates overnight. After 70% confluence was reached, the old medium was replaced with 150 pl of freshly prepared medium containing GNPs at the desired concentrations. After two hours of incubation and the cell uptakes reached about 2.96×10⁴ GNPs per cell, the medium was replaced with fresh medium. The cells were treated with or without irradiation. Cell response to the GNPs with and without irradiation was monitored by the MTT assay following the manufacturer's instructions and clonogenic survival assay. For clonogenic survival assay, the cells either with or without treatment were incubated for 2-3 weeks. Cells were then fixed with 3:1 ethanol to acetic acid solution and stained with crystal violet. Colonies were counted for the control and experimental groups, with each experiment performed in triplicate.

### Cell Irradiation

Human breast cancer MCF-7 cells and non-malignant breast cell line (MCF-10A) were used in these studies. 200 kVp X-ray irradiation with the PANTAK Therapax3 series, y-ray irradiation with the Shepherd-Mark I-68A ¹³⁷Cs Irradiator (J. L. Shepherd & Associates, San Fernando, Calif.) and the ⁶⁰Co irradiator (Atomic Energy of Canada Ltd) were used in this experiment. The targeted cells were exposed to hybrid-gold nanoparticles (Glu-GNPs or AET-GNPs) and then followed by either (i) 200 kVp X-ray irradiation at 1.I9Gy/min, (ii) γ-ray irradiation with ¹³⁷Cs at −1.57 Gy/min, or (iii) with ⁶⁰Co irradiator at −4.76 Gy/min, each with a total dose of 10 Gy at room temperature. The control experiments were performed with the same procedures as above except replacing hybrid nanoparticles with non-labeled naked GNPs. For all the experiments, after irradiation, the cells were incubated for 48 hours before measuring cell viability with the MTT assay. The cells growing in plain medium without nanoparticles or irradiation were used as a control.

### Statistical Analysis

Experimental values were determined in triplicate. All values regarding measurement and percentage of gold content were expressed as means and standard errors (SE). The one-way analysis of variance (ANOVA) and Tukey multiple comparison post-test were used. Differences less than 0.05 (p<0.05) were considered statistically significant.

### Characteristics of GNPs

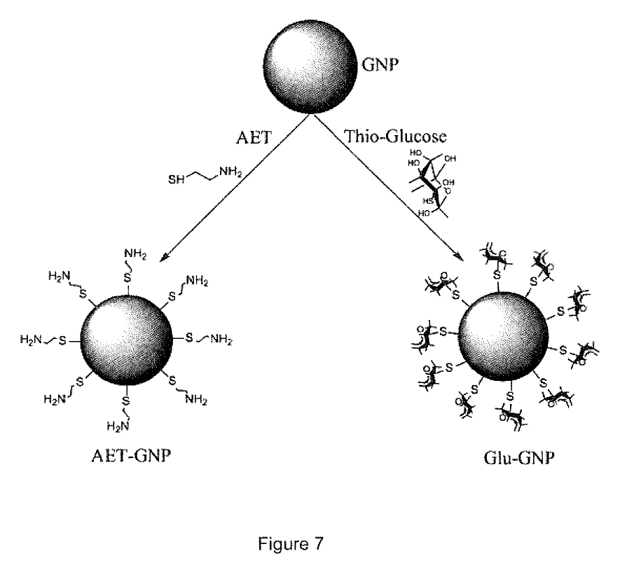

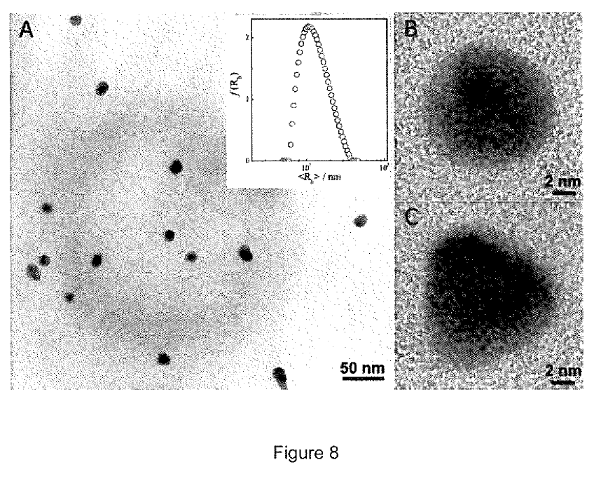

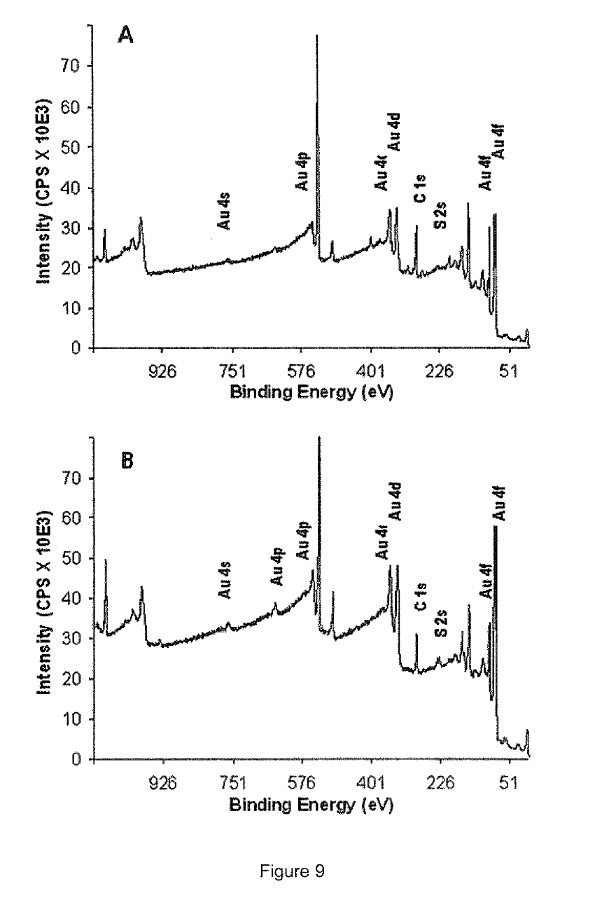

FIG. 7 shows the schematic diagram of both AET-GNPs and Glu-GNPs. FIG. 8 shows the TEM images of the gold nanoparticles. The diameters of the GNPs were measured with TEM and the average diameter of GNPs was 10.8 nm. The same size GNPs were used for all experiments in this study. To identify the binding of GNPs with either AET or thioglucose, the nanoparticles were characterized using XPS. The average numbers of bio-molecules (around 2× one nanoparticle were calculated by measuring the gold to sulfur atom ratio acquired with XPS (FIG. 9). The results are listed in Table 1.

TABLE 1
Measurement results of gold and sulfur concentration
on AET-GNP and Glu-GNP.

| | AET-GNP | Glu-GNP |
|---|---|---|
| Au (at. | 64.55 | 59.76 |
| %) | | |
| S (at. %) | 35.45 | 40.24 |

### Distribution of GNPs in Targeted Cells

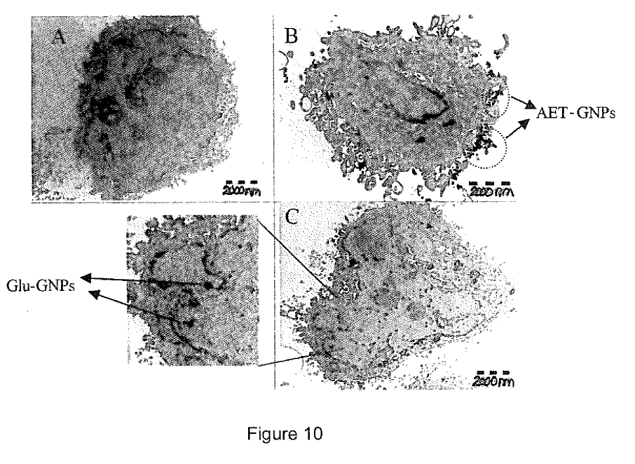

The typical distribution of GNPs in MCF-7 cells can be observed by TEM. FIG. 10A indicates the appearance of normal cells. FIG. 10C is the micrograph of the cells treated with 15 nM Glu-GNPs. The figure shows that most of GNPs were distributed in the cytoplasm. The appearance of MCF-7 cells treated with 15 nM AET-GNPs is shown in FIG. 10B, in which we can see that most GNPs were bound to the cell membrane in this case.

### GNPs Bound to or Internalized by Target Cells

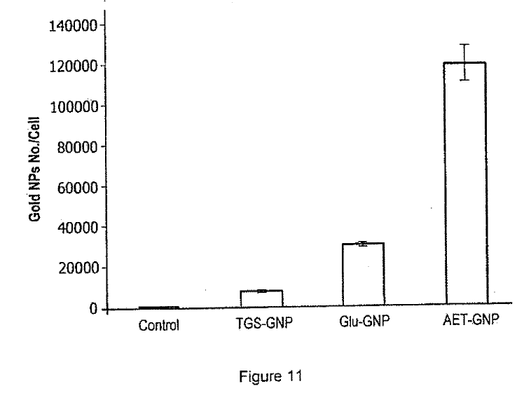

The number of GNPs that bound to or were taken up by MCF-7 cells in cell lysate was quantified with ICP-MS. After the cells were exposed to 15 nM of naked GNPs, AET-GNPs, or Glu-GNPs for two hours, the average number of GNPs associated with each MCF-7 cell was 7.34×10³ for naked GNPs, 2.96×10⁴ for Glu-GNPs, and 1.187^{x} 10⁵ for AET-GNPs per cell (FIG. 11). Exposure to AET-GNPs resulted in a factor of three to four-fold increase of GNPs compared to Glu-GNPs in each target cell. When 15 nM Glu-GNPs and 3.85 nM AET-GNPs were used to treat the cells respectively, the targeted cells have the same level of nanoparticles for each type of GNPs in the targeted cells, or about 2.96×10⁴ GNPs per cell. We later use these samples in the experiments of cytotoxicity assay and irradiation studies.

### Cytotoxicity of GNPs

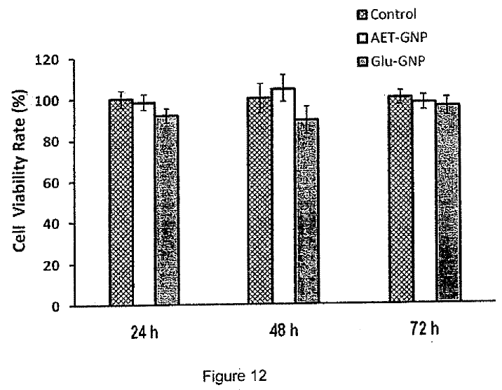

The cytotoxicity of either AET-GNPs or Glu-GNPs on MCF-7 cells that were treated for 24, 48, or 72 hours respectively was measured with the MTT assay. The data was analyzed with a t-test, and no significant difference was noticed between the control and the AET-GNP-treated cells or the Glu-GNP treated cells (p<0.05) as shown in FIG. 12. By using the percentage to measure the viability, no significant changes in the cytotoxicity were seen as incubation time increased. Therefore, we can conclude that neither the AET-GNPs nor the Glu-GNPs induced remarkable cytotoxicity in MCF-7 cells.

### Irradiation: Enhancement of Irradiation by GNPs

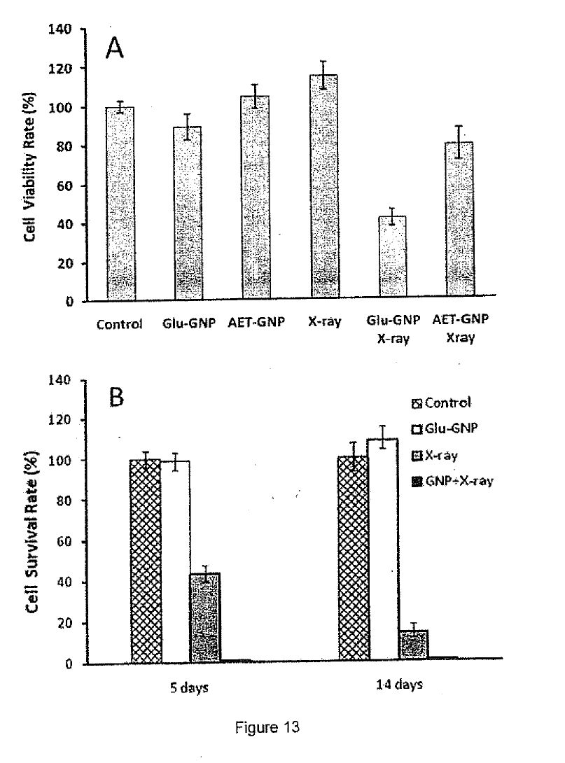

The target cells were treated with GNPs for two hours. Both the treated and untreated cells were then irradiated with 200 kVp X-rays with a dose of 10Gy. We detected the induced cytotoxicity with the MTT assay 48 hours after the irradiation. The responses of the MCF-7 cell line with and without X-ray treatment are shown in FIG. 13. After irradiation, 200 kVp stimulated cancer cell growth and induced a cell viability of 114.8% after 48 hours (FIG. 13A). Glu-GNPs (15 nM) and AET-GNPs (3.85 nM) induced about 63.5% and 31.7% increase in radiation cytotoxicity respectively, when compared to irradiation alone.

In clonogenic survival assay, 200 kVp X-rays induced a significant decrease in cell survival (FIG. 13B) after five days. However, there were still 43.2% of cancer cells surviving after five days and 13.8% of cells survived after fourteen days. For the cells exposed to Glu-GNPs, the irradiation of 200 kVp X-rays resulted in complete cancer cell death after five days and this trend continued.

### Comparison of Radiation Sensitivity in Cancer or Non-Malignant Cells

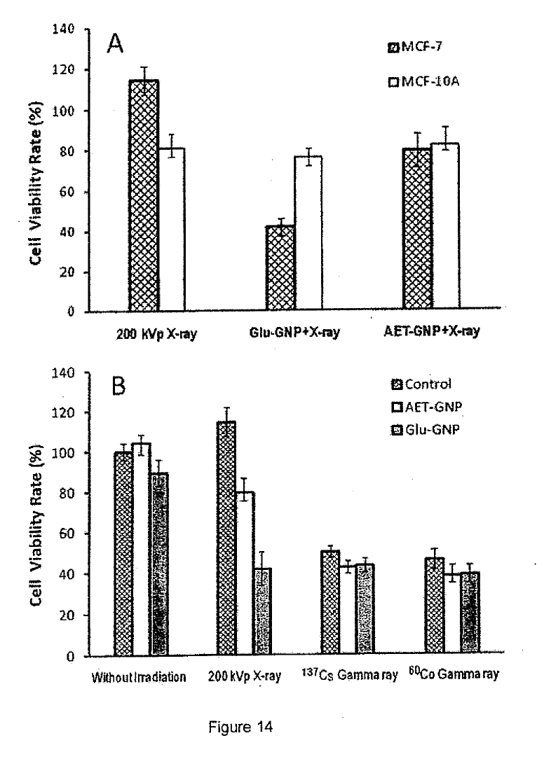

MCF-10A cells were selected as a non-malignant breast cell line to provide evidence to prove the efficiency of the functional gold nanoparticles in cancer killings. After exposing to Glu-GNPs, both MCF-7 and MCF-10A cells uptook the same level of GNPs because both types of cell lines have the same growth rates and need the same amount of glucose for metabolism (the results were not shown in this paper). FIG. 14A shows that 200 kVp X-ray induced a 20% cell viability decrease in the MCF-10A cells, but not in the MCF-7 cells. After incubation with Glu-GNPs for two hours, the cell viability of MCF-7 cells decreased to 40% after irradiation. However, no significant changes in radiation sensitivity were shown in MCF-10A cells that were treated either with or without Glu-GNPs (p<0.05). These results indicate that Glu-GNPs only enhance the radiation sensitivity in cancer cells but not in non-malignant breast cells. These results also show that the modified nanoparticles of the invention can be used for targeted cancer treatment.

Radiation Cytotoxicity Resulting from Various Types of Irradiation

The capability of GNPs to enhance the cytotoxicity induced in MCF-7 breast cancer line by various types of irradiation (X-ray and γ-ray) was evaluated. Compared to 200 kVp X-rays (30% cell death for AET-GNPs and 60% cell death for Glu-GNPs), ¹³⁷Cs γ-ray and ⁶⁰Co γ-ray have smaller enhancement on cell killing (12.7% cell death for ¹³⁷Cs γ-ray and 13.1% for ⁶⁰Co γ-ray) (FIG. 14B).

### Example Three

### Making Gold Nanoparticles bound with PET Tracer ([18F]flurodeoxyglucose)

Step 1. Creating gold-based hybrid nanoparticles.

Step 2. Binding the hybrid nanoparticles covalently to PET tracers.

Step 3. Testing cell uptake of gold-based hybrid nanoparticles.

¹⁸F-6-FDG was synthesized using radioactively labeled fluorine ion as a nucleophile for displacement of a tosyl or trifyl group at C-6 of acetyl-protected glucose.

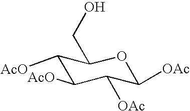

1,2,3,4-Tetra-O-acetyl-beta-D-glucopyranose

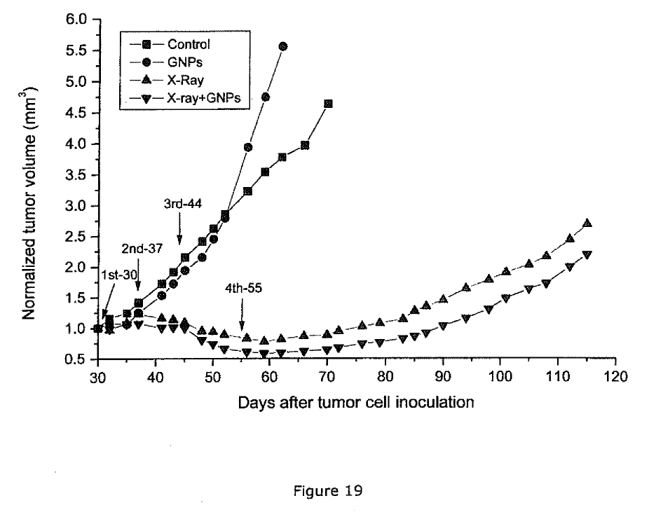

For the organic synthesis of ¹⁸F-6-FDG capped gold nanoparticles, tosylation or trifylation of the free hydroxyl group at the C-6 position of 1,2,3,4-Tetra-O-acetyl-beta-D-glucopyranose was done (shown in FIG. 19), a commercially available reagent from Sigma-Aldrich. Thioacetate was then successfully displaced the O-acetyl group at the C-1 position of the tosylate or trifylate intermediate. After displacement of the tosyl or trifyl by ¹⁸F, the radioactively labeled intermediate was hydrolyzed to give a ¹⁸F labeled thioglucose, which then binds to gold nanoparticle rapidly via a strong covalent bond between the free thiol group and gold nanoparticle, and subsequently ready for injection to patients. Both 2,3,4-Tri-O-acetyl-1-S-acetyl-1-thio-6-O-(4-methylphenylsulfonyl)-beta-D-glucopyranose and 2,3,4-Tri-O-acetyl-1-S-acetyl-1-thio-6-O-trifluoro-methanesulfonyl-beta-D-glucopyranose were synthesized, which were labeled with ¹⁸F (our final as product shown in FIG. 20).

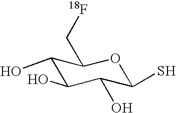

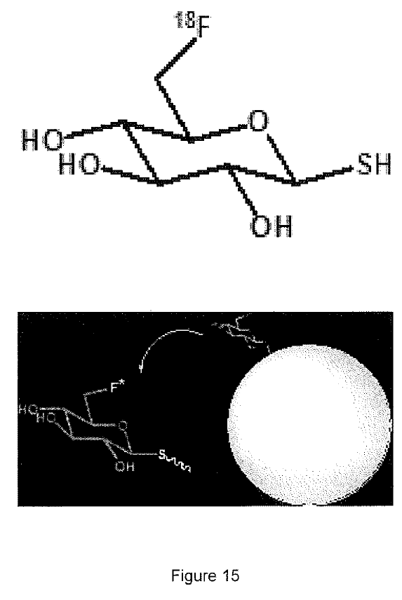

FDG with thiol group, which is ready to bind with gold nanoparticle as shown in FIG. 15.

### Example Four

### FDG-Gold Nanoparticles Used as a Sensitizer of Radiotherapy in vivo

### Mouse Preparation

Approximately 5 week-old female Balb/C nude mice were used. All mice were quarantined and acclimatized to laboratory conditions for two weeks before inoculation with tumour cells.

### Cell Preparation

MCF-7 breast cancer cells were cultured until the mice were ready for inoculation. The cells were harvested, counted, diluted in 0.9% saline solution for injection.

### Experimental Procedure

1) 200 μl of the above cell solution containing 5.0×10⁶ MCF-7 cells was injected subcutaneously into the right flank of each mouse. Tumor volume was calculated from external measurements of length (L), width (W), and height (H) three times a week after tumor cell inoculation. Calculation of tumor volumes was based on the assumption that tumors will be hemi-ellipsoids: V=L*W*H*0.5236.

2) After tumor volumes reach about 300 mm³, mice were ready for treatment and were randomly divided into 4 groups:
Group 1: Control-tumour-bearing mice;
Group 2: GNPs-tumour-bearing mice injected FDG-GNPs;
Group 3: X-Ray-tumour-bearing mice treated by x-ray irradiation; and
Group 4: X-Ray+GNPs-tumour-bearing mice treated by x-ray irradiation after FDG-GNPs injection.

3) For groups 2 and 4, two hours before FDG-GNPs injection, food was withheld from the mice. 200 uL of FDG-GNPs solution suspended in sterilized RODI water was injected into the tail vein of each mouse.

4) For groups 3 and 4, x-ray irradiation was delivered at 2 hours post-injection of FDG-GNPs (X-Ray equipment: Pantek orth-voltage, 200 Kvp, 10Gy).

5) Treatment of mice with FDG-GNPs injection and x-ray irradiation was done weekly for four straight weeks.

6) Mice were monitored daily for animal survival and morbidity and for weight loss and inactivity in addition to signs, such as scruffy appearance, listlessness, and compromised breathing during the study period. Meanwhile, tumor volumes were measured three times weekly.

### End Point Determination

Mice were sacrificed when the tumor weight in the treatment and control groups reaching 10% of the baseline body weight, or when the largest tumor diameter reached 25 mm, or when the largest tumor bi-dimensions reached 16 mm×16 mm, whichever came first.

### Data Analysis

All the data were normalized based on the tumor volume measurement prior to first treatment delivery. Data was analyzed using a one-way ANOVA test.

FDG-GNPs Sensitize Tumors to Irradiation Therapy in vivo

The difference between the group treated with irradiation only (-▴-) and the group treated with irradiation plus FDG-GNPs (-▾-) was significant (p=0.00971), which means FDG-GNPs enhance radiosensitivity of breast cancer MCF-7 cell tumors to X-Ray irradiation (200 Kvp, 10Gy) in vivo in a mouse model.

### Example Five

### Testing Safety of PET-Guided Gold Nanoparticles in an Animal Model

The acute toxicity of PET-guided gold nanoparticles will be studied in the BALB/c mouse model. The starting dose will be one-tenth of the safe dose identified in the mouse model for the phase I clinical trial, which in turn identifies human toxicity as the primary endpoint. There will be a dose-escalation schedule within the human trial. The acute toxicity of the nanoparticles will be assessed as per the Organization for Economic Co-operation and Development (OECD) guideline, with the Up-and-Down-Procedure (UDP) (#425).

The following is a detailed technical description of the in vivo test: four to five weeks old BALB/c nude mice will undergo tail-vein intravenous injection of gold nanoparticle fluid. A total of 45 animals will be used in this part of the study, 15 for PET-guided gold nanoparticles, 15 for regular gold nanoparticles, and 15 for the control group, in which saline alone will be injected intravenously. The control group would account for the effect of anesthesia and injection procedures. Gold nanoparticles at a starting concentration of 100 μg per ml will be used for each fixed volume (0.2 ml) of intravenous injection, as previous experiments showed that this dose was not toxic. On the first day, a concentration of 100 μg per ml of gold nanoparticles and PET-guided nanoparticles will be administered for one mouse from each group. The same volume of saline will be injected in the control mouse. The treated mice will be observed continuously for the first 4 hours, every hour for the following 4 hours, then every 12 hours for the following 2 days, and then daily for 2 weeks.

The animals' morbidity will be assessed using a morbidity score. The animal should be humanely euthanized with euthanyl when the score indicates that the animal has reached the end point. If the first treated animals survive the concentration of 100 μg per ml during the first 24 hours, the next concentration (1000 μg per ml) will be applied and the observations will be conducted as previously mentioned. This will provide a ten-fold dosing range to determine if the previously found concentration is truly the most appropriate dose without reaching toxic drug levels (high dose) or therapeutic insensitive doses (low dose). Upon reaching a concentration that is not successful, the dose will be decreased to the last found concentration. The previous procedures will be repeated until one of the stopping criteria is satisfied. It is expected that the reachable concentration will be over 1 g per ml with the soluble PET-guided nanoparticles.

Necropsy: All of the experimental mice will be sacrificed for necropsy. Histological examination and pathology reports will be issued by a consulting veterinarian. The liver, lung, heart, kidney and brain will be harvested from euthanized animals, and sent for examinations and nanoparticles quantification. Blood will be collected, using the cardiac bleeding technique, and sent for complete chemistry panel and blood count.

ALTHOUGH PREFERRED EMBODIMENTS OF THE INVENTION HAVE BEEN DESCRIBED HEREIN IN DETAIL, IT WILL BE UNDERSTOOD BY THOSE SKILLED IN THE ART THAT VARIATIONS MAY BE MADE THERETO WITHOUT DEPARTING FROM THE SPIRIT OF THE INVENTION OR THE SCOPE OF THE APPENDED CLAIMS.

1. A modified gold nanoparticle comprising a gold core and a surface thereon, wherein said surface comprises a modification selected from a coating of cysteamine and/or cysteamine/thioglucose.

2. The modified gold nanoparticle of claim 1, wherein said modification is cysteamine.

3. The modified gold nanoparticle of claim 1, wherein said nanoparticle is covalently bound to a PET tracer.

4. The modified gold nanoparticle of claim 3, wherein said PET tracer is an organic molecule labeled with a radionuclide selected from ¹¹carbon, ¹³nitrogen, ¹⁵oxygen and ¹⁸fluorine.

5. The modified gold nanoparticle of claim 4, wherein said PET tracer is selected from [18F]flurodeoxyglucose and [18F]fluro-17-estradiol.

6. The modified gold nanoparticle of claim 4, wherein said gold nanoparticle is solid or hollow.

7. The modified gold nanoparticle of claim 6, wherein said gold nanoparticle is hollow and farther comprises a drug.

8. The modified gold nanoparticle of claim 5, wherein said covalently bound PET tracer is [18F]flurodeoxyglucose.

9. The modified gold nanoparticle of claim 6, wherein said coating is provided as a single layer or multiple layers of said cysteamine and/or cysteamine/thioglucose.

10. The modified gold nanoparticle of claim 9, wherein the diameter of the gold core is in the range of about 0.8 to 400 nm.

11. The modified gold nanoparticle of claim 10, wherein the diameter of the gold core is in the range of about 0.8 to 20 nm.

12. The modified gold nanoparticle of claim 11, wherein the diameter of gold core is about 0.8 to 3 nm.

13. The modified gold nanoparticle of claim 11, wherein said diameter of gold core is about 10-11 nm.

14. A composition comprising the modified gold nanoparticle of claim 13.

15. A method of imaging and/or treatment of cancer in a mammnal, comprising administering to said mammal the composition of claim 14, irradiating said mammal, and detecting radiosensitivity enhancement in said mammal versus a suitable control.

16. The method of claim 15, wherein said cancer is selected from breast cancer and prostate cancer.

17. A method for enhancing the effects of radiation directed to a tissue or a population of cells in a mammal, comprising administering an amount of modified gold nanoparticles to said mammal to achieve a concentration in said tissue or said population of cells of the mammal of at least about 0.1% metal by weight; and subsequently irradiating the animal with radiation directed to said tissue or said population of cells, wherein said radiation is in the form of x-rays of about 1 keV to about 25,000 keV and wherein said modified gold nanoparticle comprises a gold core and a surface thereon, wherein said surface comprises a modification selected from a coating of cysteamine and/or cysteamine/thioglucose.

18. The method of claim 17, wherein said tissue or said population of cells is a tumor.

19. The method of claim 18, wherein said tumor is a solid tumor selected from the group consisting of carcinomas, brain tumor, melanomas, lymphomas, plasmocytoma, sarcoma, glioma and thymoma.

20. The method of claim 19, wherein said tumor is myeloma, leukemia, or a tumor of the oral cavity, pharynx, digestive system, respiratory system, bones, joints, soft tissue, skin, breast, genital system, urinary system, eye, orbit, the nervous system, or endocrine system.

21. The method of claim 20, wherein the diameter of the gold core is in the range of about 0.8 to 400 nm.

22. The method of claim 21, wherein the diameter of the gold core is in the range of about 0.8 to 20 nm.

23. The method of claim 22, wherein the diameter of gold core is about 0.8 to 3 nm.

24. The method of claim 22, wherein the diameter of gold core is about 10-11 nm.

25. The method of claim 12, wherein said modification is cysteamine.

26. The method of claim 17, wherein said gold nanoparticles are further covalently bound to a PET tracer.

27. The method of claim 26, wherein said PET tracer is selected from an organic molecule labeled with a radionuclide selected from ¹¹carbon, ¹³nitrogen, ¹⁵oxygen and ¹⁸fluorine.

28. The method of claim 27, wherein said PET tracer is selected from [18F]flurodeoxyglucose and [18F]fluro-17-estradiol.

29. The method of claim 28, wherein said PET tracer is [18F]flurodeoxyglucose.

30. The method of claim 28, wherein said gold nanoparticles are administered to said mammal by intravenous or intra-arterial injection, direct injection into said tissue or population of cells, implantation of a device capable of a slow release of said metal nanoparticles, or injection into a body cavity.

31. The method of claim 30, wherein said x-rays are in the form of microbeam arrays of x-rays.

32. A method for real time targeted cancer imaging/therapy, said method comprising administering to a mammal diagnosed with a cancer, an effective amount of a modified gold nanoparticle covalently bound to a PET-tracer and subjecting said mammal to positron emission tomography for a time effective to visualize said cancer, wherein said modified gold particle comprises a gold core and a surface thereon, said surface comprising a modification.

33. The method of claim 32, wherein said PET-tracer is selected from an organic molecule labeled with a radionuclide selected from ¹¹carbon, ¹³nitrogen, ¹⁵oxygen and ¹⁸fluorine.

34. The method of claim 33, wherein said PET tracer is selected from [18F]flurodeoxyglucose and [18F]fluro-17-estradiol.

35. The method of claim 34, wherein said PET tracer is [18F]flurodeoxyglucose.

36. The method of claim 27, wherein said modification is selected from a coating of cysteamine and/or thioglucose.

37. The method of claim 36, wherein said gold nanoparticle is solid or hollow.

38. The method of claim 37, wherein said gold nanoparticle is hollow and further comprises a drug.
